(19) United States
(12) Patent Application Publication
(10) Pub. No.: US 2002/0183829 A1
(43) Pub. Date: Dec. 5, 2002

(54) Material for medical stents and device for the intracorporeal inductive heating of these stents

(76) Inventors: Claas Doscher, Hamburg (DE); Bernhard Fuchs, Hamburg (DE)

Correspondence Address: Min (Amy) S. Xu, DORSEY & WHITNEY LLP, Suite 1500, 50 South Sixth Street, Minneapolis, MN, 55402-1498, US

(21) Appl. No.: 10/093,228

(22) Filed: Mar. 6, 2002

(30) Foreign Application Priority Data
Mar. 20, 2001 (DE) 101 13 659.5

(51) Int. Cl. A61F 2/06

(52) U.S. Cl. 623/1.15

(57) ABSTRACT

Stents are inserted into vessels to expand and splint stenoses. To treat restenosing vessels, it has been known to inductively heat the stent to a temperature at which proliferating tissue cells are killed off. Conventionally known induction heating devices have a high level of technical complexity and require a complex temperature control for the stent.

Therefore, a new metal stent is proposed which consists of a metal alloy with a relative magnetic permeability higher than 100 and a curie temperature of an order of magnitude under which any further restenosing inside the stent is inhibited or treated and above which damage to the vessel occurs.

The device has a special induction coil which is located at a defined distance from the organism.

### CROSS REFERENCE TO RELATED APPLICATIONS

The present invention is a Continuation-in-Part of German Patent Application No. 101 13 659.5, filed on Mar. 20, 2001 of which is incorporated herein by reference.

### BACKGROUND OF THE INVENTION

Stents have long been known and used as internal supports of vessels in medicine. The objective of inductively heating the stent is to prevent restenosis inside the stent.

Stents are implants made of metal or plastic which are inserted into vessels to expand and splint occluded or narrowed vessels, so-called stenoses. Accordingly, such stents are basically cylindrical and consist of a lattice-like material of various structures and thicknesses. The dimensions, the choice of material, and the lattice-like structure or wire netting of the stents are designed to ensure that they are flexible, on the one hand, and that they are able to sufficiently support the vessel whenever stress is exerted on the vessel involved, on the other hand. As a rule, the material used is a nonoxidizing steel alloy with or without coating, e.g., made of gold.

Such a stent is introduced into the vessels by means of a catheter and placed in situ into its final position by means of a balloon, thus ensuring that the stent makes contact with and sits close to the walls of the vessel.

As a result of arteriosclerotic processes, so-called in-stent restenosing, these stents become reoccluded over time so that a stenotic treatment must be carried out repeatedly at certain time intervals. To treat these restenoses, it has been known to introduce radioactively seeded catheters into the vessels, which kill the stenosing tissue. The body is subsequently able to degrade the dead cells by itself. This method of brachytherapy, however, entails risks and undesirable side effects.

German Patent No. 295 19 982.2 describes an induction heating device with a generator and an induction coil, by means of which electrical energy is transformed into magnetic energy. This magnetic energy is directed onto a metal implant, for example a stent, inside the body of the patient, for which purpose the body of the patient is pushed into an appropriately large induction coil and the implant is positioned inside and into the axial center of the induction coil. The metal implant absorbs the magnetic energy and, due to its specific properties, transforms it into thermal energy, and this thermal energy is subsequently released into its immediate environment. In this manner, the tissue cells which proliferate in the vicinity of the implant are heated to a temperature which kills these cells.

Using normal technical means, this induction heating device makes it possible to heat the stents only very slightly. But this is insufficient since, as is well known, temperatures between 42° C. and 55° C. are required to kill tissue cells. Based on the size of the induction loop and the material properties of the stent, the electrical energy required to generate such a temperature would have to be of an order of magnitude impossible to achieve with conventional generators without an excessively high degree of complexity. Special generators, on the other hand, are inordinately large, expensive and hard to handle and markedly limit the field of application. Furthermore, in the normal medical practice, generators with a consumption of more than 10 kW entail complex and expensive safety measures.

Another disadvantage is that due to the position of induction coil, the magnetic forces penetrate the human body in its longitudinal direction and therefore, as a rule, only act in the longitudinal direction on the cylindrical stents. Thus, because of its smaller contact area, the stent offers less resistance to the magnetic forces, and therefore absorbs less magnetic energy and, as a result, produces less thermal energy. Either the temperature thus generated is not sufficiently high or the electrical output energy must be increased even more, which is technically not feasible for the reasons mentioned earlier.

Still another disadvantage is that the actual temperature of the stent cannot be measured. But for the protection of the patient, temperature control is absolutely necessary.

A similar device and method for heating a stent in the human body is described in European Patent No. 1 036 574 A1. This device is equipped with an additional high-frequency oscillator and a tubular chamber which is located between the generator and the induction coil. The tubular chamber is meant to increase the electrical output energy, and the high-frequency oscillator is meant to generate an alternating magnetic flux.

These additional elements, in fact, generate a higher magnetic field and thus a higher heat output on the stent; however, the increase in temperature is still too low since only the hysteresis losses in the metal body are utilized. The degree of technical complexity required is unacceptably high and disproportionate relative to the intended goal. Furthermore, the diameter of the induction coil is designed to be so large that it can encompass an organism, which, in turn, requires an inordinately high electrical power supply.

According to an alternative solution proposed in European Patent No. 1 036 574 A1 mentioned above, the induction coil has a smaller diameter and is positioned in the transverse direction with respect to the human body. Although, on the one hand, the decreased diameter of the induction coil makes it possible to reduce the electrical energy consumption, increased electrical energy is required, on the other hand, since the stent in this position is located outside the coil and thus in an area in which the magnetic field of the induction coil is no longer very active. This, in turn, again requires additional electrical energy which the generator is not capable of producing without a disproportionately high degree of technical complexity. This device also does not allow the temperature of the stent to be limited.

Thus, the problem to be solved by the present invention is to develop a generic stent which, while retaining all technical properties, improves the ratio between the electromagnetic energy supplied and the thermal energy converted by the stent; an additional problem to be solved is to increase the efficiency of a generic device for heating the new stent. Another problem to be solved by this invention is to maintain an automatic temperature control by using a different material and by utilizing the curie effect.

### SUMMARY OF THE INVENTION

The new stent and the new device for heating a stent eliminate the prior-art drawbacks mentioned. The special advantage of the new stent is mainly that a material is used which has an increased susceptibility for the electromagnetic field strength, the prerequisite of which is a high magnetic permeability. To achieve this, yet another effect is utilized, by means of which the stent is heated as a result of the occurring eddy current losses. When the material and the design of the stent are properly chosen, the eddy currents are increased to the point that the heat absorption is considerably increased while the degree of technical complexity required is low.

If the frequency of the induced H field is increased above a characteristic and material-specific value f_{w}, the eddy currents outweigh the other effects.

$${}^{f} w = \frac{8 \cdot p}{μ \cdot {D}^{2}}$$

In this equation, p stands for the specific resistance of the material and μ stands for the product of the permeability and the relative permeability. D stands for the thickness of the material. If the permeability is high, the frequency is typically far below the normally utilized generator frequencies.

All this reduces the consumption of electrical output energy and thus the degree of technical complexity for the electrical power source. It is especially useful to ensure that the metal alloy has a permeability of more than 100. Preferably, the permeability should amount to several 1000. The metal alloy to be used preferably is a nickel-iron alloy; however, alloys of nickel and copper, nickel and palladium, palladium and cobalt, and nickel and silicon can be used as well.

Another important advantage is obtained by the fact that this metal alloy has a high curie temperature which ensures that the stent is maintained at a temperature at which the tissue proliferation is destroyed. By ensuring that the alloy is appropriately composed, it is possible to set this curie point of the material, for example, to temperatures between 40° C. and 60° C., preferably between 42° C. and 45° C. The temperature of the stent is not further increased once this curie temperature has been reached. Thus, the curie temperature is the maximum temperature that can be reached; it also prevents an overheating of the stent. This makes it possible to forgo the use of a device that controls the temperature, and the device for heating the stent can be designed simply and cost-effectively.

Another advantage is obtained if the stent is covered with an electrically highly conductive material, which ensures an improved distribution of the temperature. Yet another advantage is that the coating is corrosion-resistant. It is also useful if the outside surface of the cylindrical body that faces the vascular wall is coated with an only sparingly conductive material to ensure that the heat generated by induction in the stent flows to the inside surface of the stent where it preferably helps to disintegrate the restenosis inside the stent.

The device for heating a stent inside an organism is characterized especially by an induction coil of optimum design which has a small diameter, on the one hand, and a relatively large exit length of the magnetic field.

It is especially useful if the induction coil has five windings and if the diameter measures 30 cm.

### BRIEF DESCRIPTION OF THE DRAWINGS

FIG. 1 shows a device for heating a stent, and

FIGS. 2 through 8 show numerical simulations of a stent.

### DETAILED DESCRIPTION OF THE PREFERRED EMBODIMENT

As is well known, the purpose of a stent is to expand an occlusion or narrowing of a blood vessel and to keep such an occluded or narrowed blood vessel patent to ensure that the blood can again circulate appropriately. Such a stent comprises a cylindrical body which is made of a braided wire or cut from a tube. In the expanded state, the cylindrical body has a diameter of 1 to 14 mm (1 to 4 mm for cardiovascular stents, 4 to 8 mm for stents for peripheral vessels, 10 to 14 mm for aortic stents) and, depending on the damage to the vessel to be treated, a length from 10 to 100 mm. The thickness of the wall of the stent ranges from approximately 0.3 to 1.2 mm.

The material of a stent that can be easily heated inductively is made, e.g., of a nickel-iron alloy. The alloy components are chosen to ensure that the relative permeability of the nickel-iron alloy is approximately 100,000 and the curie temperature is approximately 50° C. to 55° C. The relative permeability is defined as the ability of the alloy to absorb optimum magnetic field energy, and the curie temperature Tc defines the transformation temperature above which spontaneous magnetization disappears. It separates the disordered paramagnetic phase at T>Tc from the ordered ferromagnetic phase at T<Tc.

A stent made of this alloy can be covered with a gold coating or another coating to ensure that the overall device is corrosion-resistant and highly conductive.

Furthermore, the dimensions of the cylindrical body, the design of the sectional shape, and the wall thickness are chosen to ensure that the stent can be radially compressed as much as possible so as to facilitate the invasive insertion through the vessels and that it can expand once it has reached the site of stent placement, on the one hand, and that it is stable enough to ensure that it can provide the blood vessel with a sufficiently high stability.

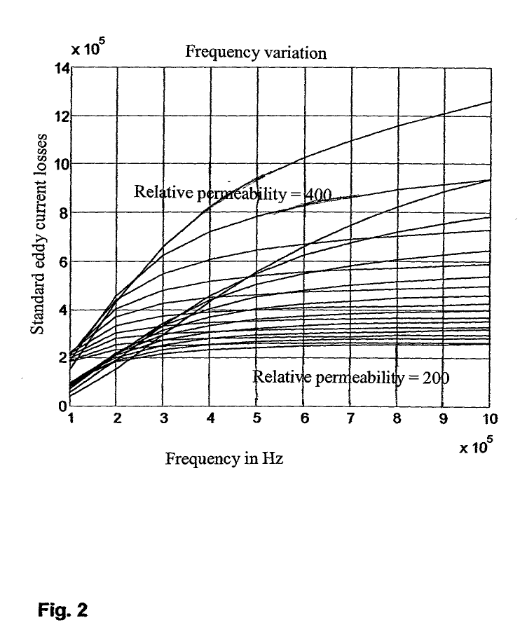

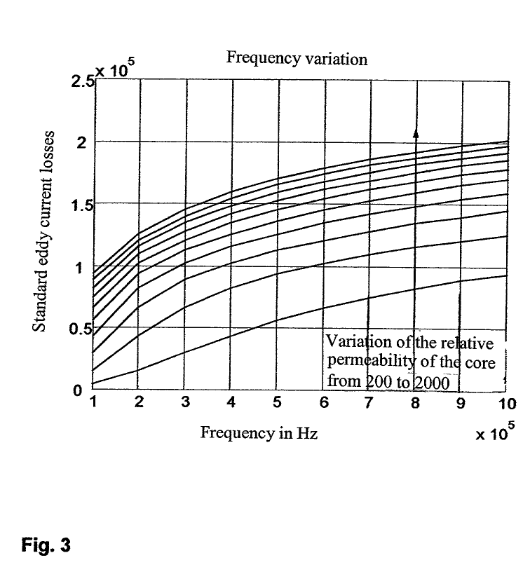

The simulation of a stent with a core and a heat-conducting gold coating as shown in FIGS. 2 through 8 was based on the following assumption. The coat coating was varied in steps of 0.5 μm up to a thickness of 5 μm. The frequency used ranged from 100 kHz to 1 MHz. The relative magnetic permeability ranged from 1 to 2000. In all simulations, the parameter tested was the relative coupled-in heat output and the loss due to eddy currents. In FIG. 2, the eddy current losses are plotted against the frequency of excitation. In FIG. 3, the thickness of the coating is 0.5 μm.

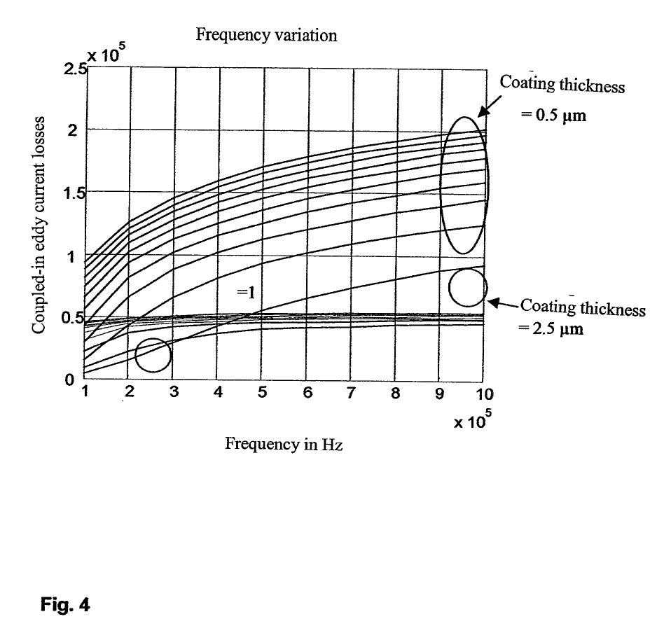

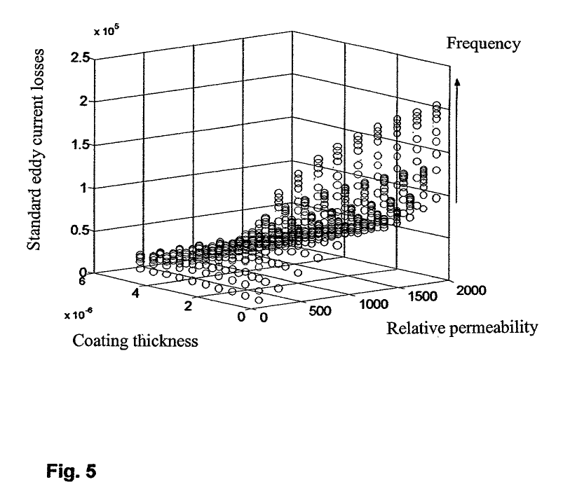

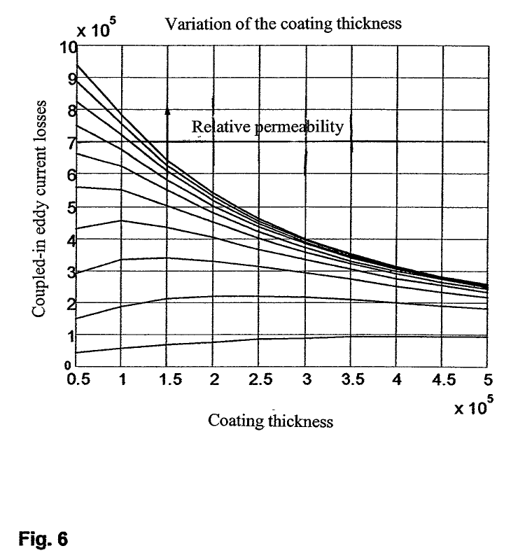

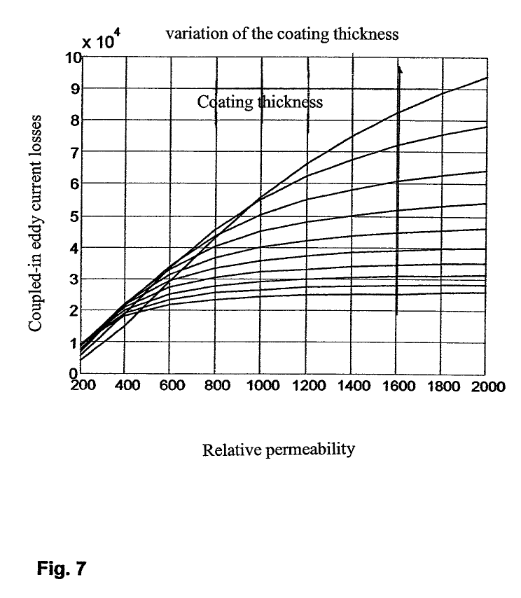

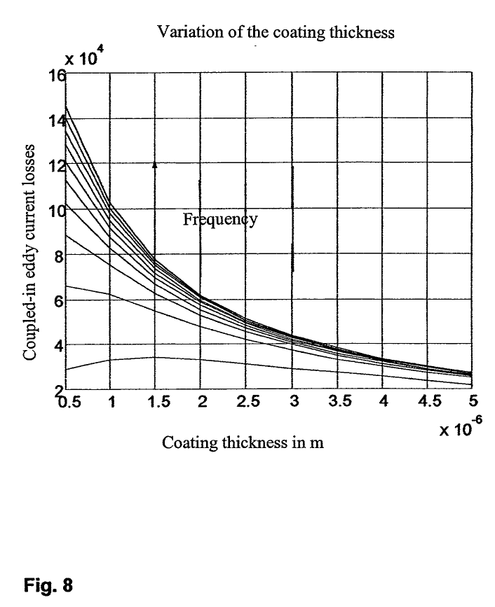

In FIG. 4, two sets of curves of increasing permeability for a coating thickness of 0.5 μm and 2.5 μm, respectively, are shown. FIG. 5 shows the coupled-in output as a function of the coating thickness, of the permeability, and of the frequency. The maximum is seen when the coating is thinnest (0.5 μm) and the permeability (2000) and frequency (1 MHz) are highest. The minimum and the maximum differ by a factor of 45, and compared to pure steel without coating, by a factor of 200,000. In FIG. 6, the coating thickness was varied while the frequency and the permeability remained constant. FIG. 7 shows variations of the thickness of the coating. The location of the maximum of the coupled-in eddy current losses also depends on the relative permeability. Above a value of 1000, the thickness of the coating should be lower than 0.5 μm. The graph in FIG. 8 is similar to the graph in FIG. 7. It results when the frequency is varied relative to the thickness of the coating. Again, at a specific frequency, a maximum is obtained at a characteristic coating thickness. At higher frequencies, the thickness of the coating can be lower than 0.5 μm.

Thus, an extremely conductive thin coating around a core with a high permeability always improves the heat output. The heat output is generated mainly in the coating. The thickness of the gold coating (a steel coating is also feasible) depends on the excitation frequency selected and on the permeability of the core. At a relative permeability of several thousand, the gold coating should preferably have a thickness of less than 0.5 μm if the core has a diameter of only 90 μm. High excitation frequencies (>500 kHz) also require a very thin coating (<0.5 μm).

The curie effect is indirectly included in the variation of the permeability. The permeability decreases at higher temperatures. The permeability as plotted against the temperature is again dependent on the material. Depending on the permeability value in the normal state and after heating, the output can decrease by factors up to several 100,000.

In principle, the following ferromagnetic materials can be used as starting materials for the method described in this invention:

| Name of the material | Curie temperature in ° C. |
|---|---|
| Cobalt in pure form | 1130 |
| Dysprosium in pure form | −168 |
| Iron in pure form | 770 |
| Gadolinium in pure form | 16 |
| Nickel in pure form | 385 |

To develop a stent with a defined curie temperature, an alloy is produced from a ferromagnetic and a nonferromagnetic material so that the curie temperature, in accordance with the mixing ratio, is lower than that of the pure ferromagnetic material.

The alloys can be:

Nickel-copper alloys

The following table presents a summary of the nickel-copper alloys:

| Name | | Curie | | |
|---|---|---|---|---|
| of the | Manufacturer of | tempera- | Frequency | |
| material | the material | ture ° C. | used | Biocompatibility |
| Ni 28% | Ames Laboratory, | 60 | 100 kHz | Dubious, coating |
| Cu | Materials | | | required, |
| | Preparation | | | intravital |
| | Center, Ames, | | | corrosion |
| | IA, USA | | | |
| Ni 29.6% | | 50 | | Dubious, coating |
| Cu | | | | required, |
| | | | | intravital |
| | | | | corrosion |
| Ni 29.6% | | 50 | 90 kHz | Dubious, coating |
| Cu | | | | required, |
| | | | | intravital |
| | | | | corrosion |
| Ni 29% | Ames Laboratory, | 60 | 100 kHz | Object of cited |
| Cu | Materials | | | test: |
| | Preparation | | | corrosion |
| | Center, Ames, | | | |
| | IA, USA | | | |

Nickel-palladium alloys

| Name of the material | Curie temperature ° C. | Biocompatibility |
|---|---|---|
| NiPd in variable | 43-58 | No data given |
| composition | | |

Palladium-cobalt alloys

| Name of the material | Curie temperature ° C. | Biocompatibility |
|---|---|---|
| Pd 6.15% Co | 50 | Probable |

The reason that this alloy is very interesting is that in addition to having ferromagnetic properties, it also practically acts like pure palladium. The most outstanding of all of the material properties is the extraordinary resistance to corrosion in a very wide pH spectrum. Palladium alloys have long been used in dentistry for the production of permanent oral implants; thus, in addition to the biocompatibility of palladium, the capacity of the alloy to withstand very high mechanical stresses has also been clinically confirmed (overview in reference [1]). In addition, since its clinical introduction in 1986, extensive clinical experience has been gathered in brachytherapy with radioactive ¹⁰³Pd implants for the treatment of prostate cancer. With the PdCo alloy mentioned above, it is possible to reach a curie temperature of 50° C. in vitro and in calorimetric experiments.

Nickel-iron alloys

The biocompatibility is the result of the gold coating. In a tissue simulated with cellulose and a controlled flow of water, it was possible to maintain a stable curie temperature of 50° C. at different water flow rates.

Nickel-silicon alloys

| Name of the material | Curie temperature ° C. | Biocompatibility |
|---|---|---|
| Ni 4% Si | 45-60 | Cytotoxic, coating |
| | | required, intravital |
| | | corrosion possible |

Both in vitro and in vivo data relating to NiSi thermoseeds are available. The pure uncoated NiSi alloys are highly cytotoxic both in vitro and in vivo, which makes a coating, e.g., in the form of plastic catheters absolutely necessary. Furthermore, during the production, so-called dendritic arms form, which, although they can be reduced by using a complex and expensive production process, have a negative influence on the ferromagnetic properties. In addition, the processes for the reduction of the dendritic arms lead to considerable irregularities in the surface, which in turn could lead to a considerable thrombogenicity if the alloy were to be used intravascularly.

Other materials used as stent materials

| Name of the material | Curie temperature ° C. | Biocompatibility |
|---|---|---|
| Bone Cement | 50-60 | No data given |
| Ferromagnetic ceramic | 43.5 | No data given |
| glass | | |

As a reaction to the local heating of cells, heat shock proteins form, which proteins cause the cells to develop a tolerance to the repeated exposure to heat. It takes the cells which, as a result, have become thermotolerant approximately 100 h to again become thermosensitive. Even if heated only for 2-3 h at 42° C., individual cells develop thermotolerances.

When intradiscal antennas within intervertebral disks were used for thermal alterations, a thermocoagulation of unmyelinated nociceptive fibers was seen at temperatures>42° C. In many cases, a reinnervation was subsequently observed.

At temperatures between 60° C. and 80° C., collagen contractions on the molecular level occur (hydrogen bonds were broken supporting the triple helix structure of the collagen molecule). Mitchel et al. also obtained these results in a swine model. At temperatures above 60° C., they observed medial necrosis, narrowing of the arterial wall, and alterations of the elastic fibers. At such temperatures, the killed-off cells are damaged as a result of direct thermal conduction. At temperatures above 80° C., vascular complications were observed in newborn lambs during balloon angioplasty at a high frequency.

In conclusion, it can be stated that a desirable target temperature of 43° C. to 60° C., and sometimes even up to 70° C., is necessary. It can, however, not be described precisely by means of which effects the desired effect of reduced restenosing is reached.

Based on these statements which are made mainly on the basis of tests involving angioplasty, one can theorize that slightly lower temperatures can be used for the inductive heating of the stent since the stent is located directly in the target cells rather than having to be pushed against them from the inside, as is the case in angioplasty.

Preliminary tests at high temperatures proved ineffective and led to an undesirably high level of damage to the vessels and the surrounding tissue. Lower temperatures, on the other hand, led to the desired effect.

A stent temperature of 46° C. for a duration of 1 or 2 min has the same effect as a stent temperature of 43° C. for approximately 20 to 25 min.

The term hyperthermia is defined as a temperature higher than 41.4° C. in the human body since at such a temperature the physiological limits of effective counterregulation are exceeded.

For this reason, the target temperature striven for should definitely be above the mentioned temperature of 41.4° C.

Since arteries of cadavers subjected to laser treatments are perforated beginning at a temperature of 76° C., the target range should be below that temperature.

There is reason to believe that there is a correlation between the development and progression of the thermotolerance of cells and the induction and accumulation of heat shock proteins.

The mucous membrane of the gastrointestinal tract is highly thermosensitive.

The heat shock proteins include HSP 27, 47, 70, 71, 90.

HSP 70 is induced by heat and reduces neointimal hyperplasias; temperatures lower than 43° C. seem not to have an effect, and temperatures above 60° C. have unacceptable effects even if the exposure time is very short. The targeted temperature range should therefore be between 43° C. and 60° C. and should not be exceeded.

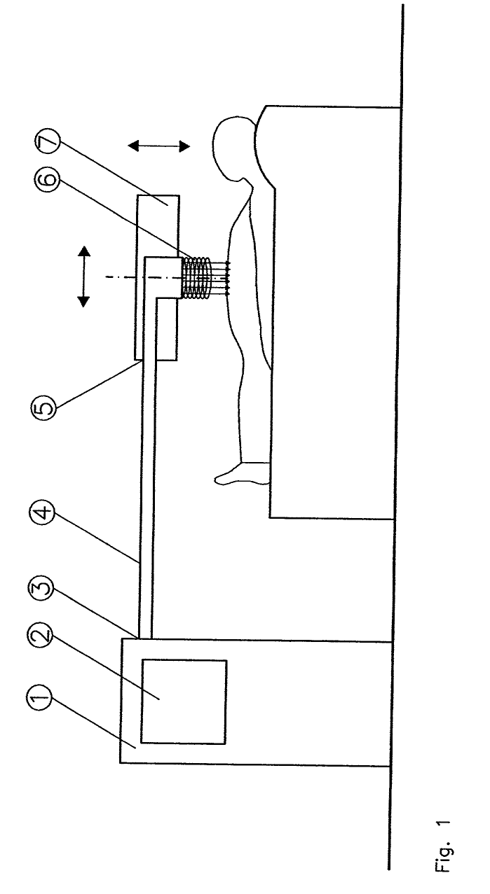

A device for heating according to FIG. 1 comprises a supply unit 1 for electrical energy which is not described in detail, with an operating and monitoring station 2 and a plug-in connection 3 for the electrical power output. Electrical cables 4 connect the plug-in connection 3 of supply unit 1 with a plug-in connection 5 for the electrical power input of an induction coil 6.

This induction coil 6 is supported by a portable unit 7 which is linearly movable in all vertical and horizontal directions and which can be rotated and swiveled around the horizontal center axis. Given these degrees of freedom, induction coil 6 can be oriented at a defined distance with respect to any location of a stent in an organism.

Induction coil 6 is attached to the underside of portable unit 7, and the axis of the coil is aligned on a vertical axis of portable unit 7. The design of induction coil 6 is such that plug-in connection 5 for the electrical power input is located on one side of movable unit 7 and that the opposite side is designed to serve as a front surface for a contactless contact with the patient.

Induction coil 6 has approximately five windings made from a copper tube which are designed so that the south pole which characterizes the entrance of the magnetic field lines is positioned on the side facing portable unit 7, and the north pole which characterizes the exit of the magnetic field lines is located on the patient side. This results in a continuous magnetic flux from portable unit 7 into the direction toward the patient. The diameter of induction coil 6 is approximately 30 cm. Thus, the induction coil has an inductivity of 32 μF, an oscillation frequency of approximately 210 kHz, and a capacity of 17.5 nF. The electrical current intensity is 15 A, and the electrical voltage is approximately 600 V.

Such an electrical supply unit 1 can be easily constructed. A device for heating a stent with this type of supply unit 1 and with such an induction coil 6 produces a focused magnetic flux which, outside induction coil 6, has an axial expansion of approximately 15 cm in the axial center and on the north pole end. The radius of induction coil 6 and the axial expansion of the magnetic flux have a ratio of approximately 1 to 1. This linear function permits an enlargement of the axial exit length of the magnetic flux but this also requires an enlargement of induction coil 6 and thus an increase in the electrical power output of supply unit 1. But there are technical limits to this. An axial exit length of approximately 15 cm, however, suffices to reach any possible location of a stent in the human body.

Although the present invention has been described with reference to preferred embodiments, persons skilled in the art will recognize that changes may be made in form and detail without departing from the spirit and scope of the invention. For example, it is appreciated that the induction coil can be any suitable signal sending antenna, and that the induction coil may have one to five or more windings.

1. Medical stent made of metal, wherein the material of the stent is a metal alloy which has a relative magnetic permeability higher than 100 and a curie temperature which is on the order of a limiting temperature under which any further restenosing inside the stent is treated and above which damage to a vessel occurs.

2. The metal stent as claimed in claim 1, wherein the metal alloy has a relative permeability of 100,000.

3. The metal stent as claimed in claim 1, wherein the material is an alloy containing a material selected from nickel, cobalt, dysprosium, iron, and gadolinium.

4. The metal stent as claimed in claim 1, wherein the alloy is a material selected from nickel-copper, nickel-palladium, palladium-cobalt, nickel-silicon, and Fe3O4 [sic].

5. The metal stent as claimed in claim 1, wherein the stent has a coating made of a material with a high electrical conductivity.

6. The metal stent as claimed in claim 5, wherein the coating of the stent is made of a precious metal.

7. The metal stent as claimed in claim 1, wherein a stent's outer surface which faces a wall of the vessel is coated with a sparingly heat-conducting material.

8. The metal stent as claimed in claim 1, wherein the curie temperature of the metal alloy is higher than 37° C.

9. The metal stent as claimed in claim 1, wherein the curie temperature of the metal alloy is in a range from 42° C. to 45° C.

10. A device for heating a metal stent, comprising:
an electrical supply unit; and
a portable unit with an induction coil, wherein the induction coil has at least one winding and is positioned at a defined axial distance from a stent located inside an organism;
wherein the north pole of the induction coil is directed toward the organism and ratio between an axial distance between the north pole of the induction coil and the stent in the organism and a radius of the induction coil is approximately one to one.

11. The device as claimed in claim 10, wherein the induction coil has five windings and a diameter of 30 cm.

12. The device as claimed in claim 10, wherein the metal stent is a metal alloy which has a relative magnetic permeability higher than 100 and a curie temperature which is on the order of a limiting temperature under which any further restenosing inside the stent is treated and above which damage to a vessel occurs.

13. The device as claimed in claim 12, wherein the metal alloy has a relative permeability of 100,000.

14. The device as claimed in claim 12, wherein the material is an alloy containing a material selected from nickel, cobalt, dysprosium, iron and gadolinium.

15. The device as claimed in claim 12, wherein the alloy is a material selected from nickel-copper, nickel-palladium, palladium-cobalt, nickel-silicon and Fe3O4.

16. The device as claimed in claim 12, wherein the stent has a coating made of a material with a high electrical conductivity.

17. The device as claimed in claim 16, wherein the coating of the stent is made of a precious metal.

18. The device as claimed in claim 12, wherein a stent's outer surface which faces a wall of the vessel is coated with a sparingly heat-conducted material.

19. The device as claimed in claim 12, wherein the curie temperature of the metal alloy is higher than 37° C.

20. The device as claimed in claim 12, wherein the curie temperature of the metal alloy is in a range from 40° C. to 45°C.
